(19) United States
(12) Patent Application Publication
(10) Pub. No.: US 2005/0107819 A1
(43) Pub. Date: May 19, 2005

(54) Catheter with a sectional guidewire shaft

(75) Inventor: Ghaleb A. Sater, Lynnfield, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc.

(21) Appl. No.: 10/712,374

(22) Filed: Nov. 14, 2003

(51) Int. Cl. A61M 29/00

(52) U.S. Cl. 606/194; 604/103.04; 604/528; 623/1.11

(57) ABSTRACT

A catheter for use in a body lumen including a guidewire shaft having a guidewire shaft sectional portion. The guidewire shaft sectional portion is disposed along a proximal and/or distal portion of an elongate catheter shaft for holding a guidewire in place thereon. The guidewire shaft sectional portion includes at least two clips or segments along the catheter shaft that are separated by a gap. The clips hold the guidewire in place along the catheter, and the gap between adjacent clips allows a clinician ready access to the guidewire.

### BACKGROUND OF THE INVENTION

1. Field of the Invention

The present invention relates to a medical device. More specifically, the invention relates to a catheter with a full-length guidewire shaft wherein a proximal portion of the guidewire shaft is formed from a plurality of independent segments, or clips, that allow access to a guidewire there between.

2. Background Art

Cardiovascular disease, including atherosclerosis, is the leading cause of death in the U.S. One method for treating atherosclerosis and other forms of coronary narrowing is percutaneous transluminal coronary angioplasty, commonly referred to as “angioplasty” or “PTCA”. The objective in angioplasty is to enlarge the lumen of the affected coronary artery by radial hydraulic expansion. The procedure is accomplished by inflating a balloon of a balloon catheter within the narrowed lumen of the coronary artery. Radial expansion of the coronary artery occurs in several different dimensions, and is related to the nature of the plaque. Soft, fatty plaque deposits are flattened by the balloon, while hardened deposits are cracked and split to enlarge the body lumen.

One or multiple dilations may be necessary to effectively dilate the artery. In many instances, successive dilations using a succession of balloon catheters with balloons of increasingly larger diameters may be required. In order to accomplish the multiple dilations, the original catheter must be removed and a second balloon catheter tracked to the lesion. When catheter exchange is desired, it is advantageous to leave the guidewire in place while the first catheter is removed in order to insert the second catheter without having to reestablish the path by inserting a new guidewire. To remove a balloon catheter while leaving the guidewire in place, there must be a portion of the guidewire extending out of the balloon catheter at the proximal end so that the guidewire can be held in place while the balloon catheter is removed.

Two types of catheters commonly used in angioplasty procedures are referred to as over-the-wire (OTW) catheters and rapid exchange (RX) catheters. A third type of catheter with preferred features of both OTW and RX catheters, that is sold under the trademarks MULTI-EXCHANGE, ZIPPER MX, ZIPPER, and/or MX is discussed below. An OTW catheter's guidewire lumen runs substantially the entire length of the catheter and is attached to, or enveloped within, an inflation shaft. Thus, the entire length of an OTW catheter is tracked over a guidewire during a PTCA procedure. A RX catheter, on the other hand, has a guidewire shaft that extends within only the distalmost portion of the catheter. Thus, during a PTCA procedure only the distalmost portion of a rapid exchange catheter is tracked over a guidewire.

If a catheter exchange is required while using a standard OTW catheter, the user must add an extension onto the proximal end of the guidewire to maintain control of the guidewire, slide the catheter off of the extended guidewire, slide the new catheter onto the guidewire and track back into position. Multiple operators are required to hold the extended guidewire and maintain its sterility while the catheter is exchanged.

A RX catheter avoids the need for multiple operators when changing out the catheter and therefore is often referred to as a “single operator” catheter. With a rapid exchange catheter, the guidewire is outside the shaft of the catheter for all but the distalmost portion of the catheter. The guidewire can be held in place without an extension when the catheter is removed from the body. Once the original catheter is removed, a subsequent catheter may be threaded onto the in-dwelling guidewire and tracked to the lesion. However, one problem associated with RX catheters is that the exposed portion of the guidewire may become tangled with the catheter shaft during use.

A balloon catheter capable of fast and simple catheter exchange is particularly advantageous. A catheter designed to address this need is sold by Medtronic Vascular, Inc. of Santa Rosa, Calif. under the trademarks MULTI-EXCHANGE, ZIPPER MX, ZIPPER and/or MX (hereinafter referred to as the “MX catheter”). An MX catheter is disclosed in U.S. Pat. No. 4,988,356 to Crittenden et al., and in co-pending U.S. patent application Ser. No. 10/116,234, filed Apr. 4, 2002, both of which are incorporated in their entirety herein by reference thereto.

The MX catheter includes a catheter shaft having a cut that extends longitudinally along the catheter shaft and that extends radially from a guidewire lumen to an outer surface of a catheter shaft. A guide member through which the shaft is slidably coupled cooperates with the cut such that a guidewire may extend transversely into or out of the guidewire lumen at any location along the cut's length. By moving the shaft with respect to the guide member, the effective over-the-wire length of the MX catheter is adjustable.

It is among the general objects of the present invention to provide an alternative catheter design which also allows for simple catheter exchange. What is needed is a catheter which allows for single operator catheter exchange without the use of a guidewire extension. Accordingly, the present invention is a catheter that includes a guidewire shaft with a sectional portion comprised of a plurality of independent segments, or clips. The clips of the sectional portion of the guidewire shaft secure a guidewire along a proximal or distal portion of the catheter while the catheter is being tracked in vivo and allow access to the guidewire, from between adjacent clips, during catheter loading and unloading.

### BRIEF SUMMARY OF THE INVENTION

To achieve the foregoing and other objects, and in accordance with the purposes of the present invention as embodied and broadly described herein, the catheter of the present invention provides a catheter capable of catheter exchange without the use of an exchange guidewire while reducing the chance of tangling the guidewire with the inflation shaft. A catheter according to the present invention is comprised of an inflation shaft with an inflation lumen, and a full-length guidewire shaft with a proximal portion comprised of a plurality of independent segments, or clips that hold a guidewire in place along a proximal portion of the catheter and allow access to the guidewire along the proximal portion of the catheter from between adjacent clips.

The present invention can form the basis of a stent delivery system, an angioplasty catheter, or an aspiration catheter but is not so limited. In the present invention, the catheter has a sectional proximal guidewire shaft that holds the guidewire in place adjacent a portion of the catheter shaft. The sectional shaft consists of at least two segments or clips, viz., a proximal clip located adjacent a proximal end of the guidewire shaft and a distal clip that in certain applications may be located adjacent a transition area of the guidewire shaft. In another embodiment, the sectional guidewire shaft may contain intermediate clips between the proximal and distal clips. The number of intermediate clips varies according to the length of the catheter, and any number of the intermediate clips may include a slit to allow for ease of guidewire loading and unloading.

The clips are attached along a proximal and/or distal portion of the catheter shaft. The clips allow access to the guidewire there between, such that in certain applications the guidewire is accessible and controllable during a catheter exchange without use of an extension wire.

In one embodiment, the catheter's distal shaft portion is of a relatively short length and has a guidewire shaft that is attached to, or enveloped within, a distal portion of an inflation shaft in a manner known in the art. The inflation shaft includes an inflation hub at its proximal end that is capable of fluid communication with a source of inflation fluid. Further, a balloon is mounted at a distal end of the inflation shaft to be in fluid communication with the inflation lumen. The balloon can be of any shape or size customarily used in angioplasty procedures. The inflation fluid is fluidly communicated to the balloon via the inflation lumen so that the balloon may be inflated and deflated.

Unlike standard OTW catheters, the present invention allows for catheter exchange without the use of an extension wire. With the present invention the user may slide the catheter proximally while maintaining control of the guidewire in between adjacent clips. The catheter can slide proximally until a distal tip of the catheter exits the body and control of the guidewire may be gained distal to the catheter tip until the catheter is fully removed from the guidewire. A new catheter may then be slid over the indwelling guidewire.

In an embodiment of the invention, the sectional clip portion of the guidewire shaft also holds the guidewire in place along the proximal portion of the catheter shaft. Therefore, although the guidewire is readily accessible along the catheter, the guidewire is not completely detached therefrom. Thus, unlike standard RX catheters, the guidewire shaft sectional portion reduces the chance of tangling of the guidewire with the catheter inflation shaft and improves tracking of the catheter over the guidewire.

### BRIEF DESCRIPTION OF THE DRAWINGS/FIGURES

The foregoing and other features and advantages of the invention will be apparent from the following, more particular description of a preferred embodiment of the invention, as illustrated in the accompanying drawings. The drawings are not to scale.

FIG. 1 is a side elevational view of a stent delivery system incorporating the present invention.

FIG. 2 is an enlarged view of the sectional portion of the catheter of FIG. 1.

FIG. 2A is a cross-sectional view along line I-I of FIG. 2 in accordance with an embodiment of the present invention.

FIG. 2B is a cross-sectional view along line I-I of FIG. 2 in accordance with another embodiment of the present invention.

FIG. 2C is a cross-sectional view along line I-I of FIG. 2 in accordance with another embodiment of the present invention.

FIG. 2D is a cross-sectional view along line I-I of FIG. 2 in accordance with another embodiment of the present invention.

FIG. 3 is a sectional view along line A-A of FIG. 1 in accordance with an embodiment of the present invention.

FIG. 4 is a sectional view along line A-A of FIG. 1 in accordance with another embodiment of the present invention.

FIG. 5 is a sectional view along line A-A of FIG. 1 in accordance with another embodiment of the present invention.

FIG. 6 is a cross-sectional view of a catheter in accordance with an embodiment of the present invention taken along line B-B of FIG. 1.

FIG. 7 is a cross-sectional view of a catheter in accordance with another embodiment of the present invention taken along line B-B of FIG. 1.

FIG. 8 is a side elevational view of an aspiration catheter incorporating the present invention.

### DETAILED DESCRIPTION OF THE INVENTION

A preferred embodiment of the present invention is now described with reference to the figures, where like reference numbers indicate identical or functionally similar elements. Also in the figures, the left most digit of each reference number corresponds to the figure in which the reference number is first used. While specific configurations and arrangements are discussed, it should be understood that this is done for illustrative purposes only. A person skilled in the relevant art will recognize that other configurations and arrangements can be used without departing from the spirit and scope of the invention.

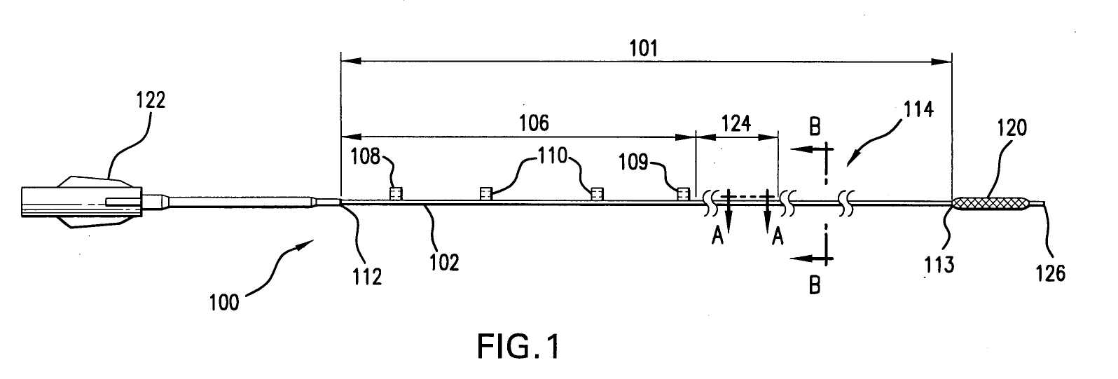

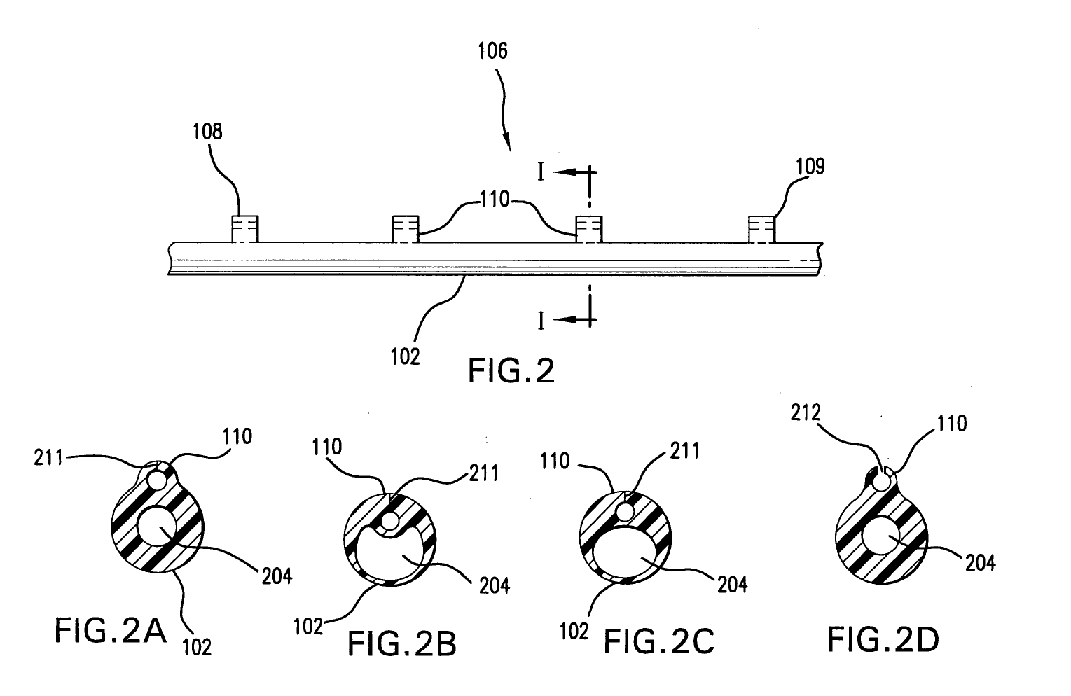

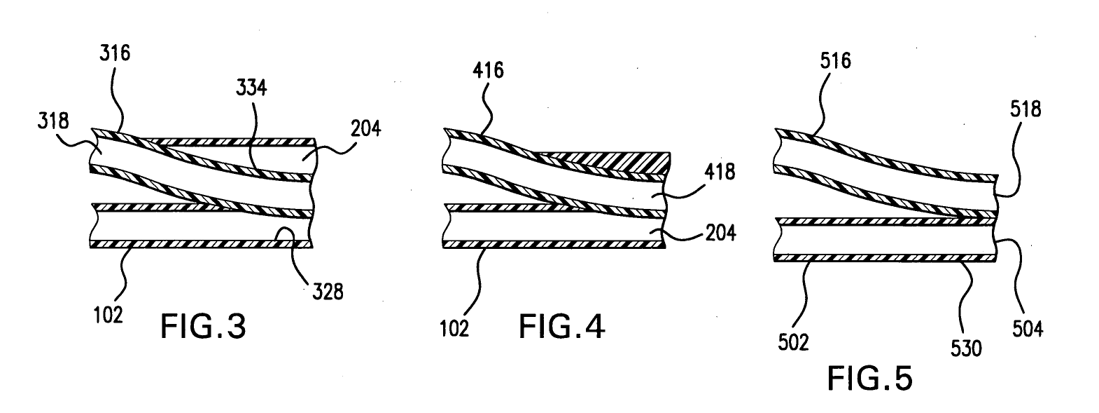

Referring to FIGS. 1, 2, and 2A-2D, an embodiment of the present invention is shown with respect to a catheter 100. In FIG. 1, catheter 100 is shown as a stent delivery system, but the present invention is not so limited and may be used on a catheter for use in PTCA, vascular drug delivery, aspiration of a treatment site, and/or other diagnostic or therapeutic procedures. Catheter 100 is provided with a dual lumen catheter shaft 101 having a proximal end 112 and a distal end 113. Catheter shaft 101 includes an inflation shaft 102, a full-length guidewire shaft having a guidewire shaft sectional portion 106 and a transition section 124. Inflation shaft 102 runs substantially the entire length of catheter 100 and has an inflation lumen 204 there through. Guidewire shaft sectional portion 106 extends from proximal end 112 of catheter shaft 101 to just proximal of transition section 124. In a distal portion 114 of catheter shaft 101 for a relatively short length, a distal portion 316, 416, 516 of the guidewire shaft may be attached to, or enveloped within, inflation shaft 102, as shown in FIGS. 3-5.

In the embodiment shown in FIGS. 1 and 2, a balloon 120 is bonded to distal end 113 of catheter shaft 101, and an interior of balloon 120 is in fluid communication with inflation lumen 204. A proximal end of inflation lumen 204 of inflation shaft 102 is in fluid communication with an inflation hub 122 that allows inflation means (not shown) to be connected thereto for inflation and deflation of balloon 120.

FIG. 2 is an enlarged view of guidewire shaft sectional portion 106 of the present invention. Guidewire shaft sectional portion 106 holds a guidewire (not shown) in place along a proximal portion of catheter shaft 101. Guidewire shaft sectional portion 106 consists of at least two clips, a proximal clip 108 and a distal clip 109. In addition, guidewire shaft sectional portion 106 may include a plurality of intermediate clips 110 between proximal clip 108 and distal clip 109. The number of intermediate clips varies depending upon the length of catheter 100. The proximal, distal and intermediate clips of guidewire shaft sectional portion 106 are disposed along inflation shaft 102 between proximal end 112 of catheter shaft 101 to just proximal of transition section 124, and in one embodiment are evenly spaced. In addition, intermediate clip 110 may include a slit 211, as shown in the embodiments of FIGS. 2A, 2B and 2C, or a slot 212, as shown in the embodiment of FIG. 2D, to allow a guidewire to pass there through, thereby allowing a clinician the option of transversely loading and unloading a catheter at the intermediate clip.

Guidewire shaft sectional portion 106 allows access to the guidewire along the proximal portion of catheter shaft 101, such that the guidewire can be manually held in place without an extension wire when catheter 100 is removed from the body. Thus, a catheter incorporating the present invention simplifies a catheter exchange because it eliminates the need for a clinician to use an extension wire. Guidewire shaft sectional portion 106 allows the clinician to slide catheter 100 proximally while maintaining control of the guidewire in between the clips of guidewire shaft sectional portion 106. Catheter 100 is slid proximally until a distal tip 126 of catheter 100 exits the body and catheter 100 is fully removed from the guidewire. A new catheter may then be slid over the indwelling guidewire.

Guidewire shaft sectional portion 106 holds the guidewire in place by acting as a path for the guidewire to follow along the proximal portion of catheter shaft 101. Therefore, although the guidewire is external to an “interior” of catheter 100, the guidewire is not completely free from the proximal portion of catheter shaft 101, as for instance it would customarily be in a RX catheter. Thus, guidewire shaft sectional portion 106 reduces the chance that the guidewire will become entangled with the proximal portion of the inflation shaft 102, thereby improving tracking of catheter 100 over the guidewire.

As discussed with reference to FIG. 1, catheter shaft 101 includes transition section 124, which is a section of the catheter shaft where the guidewire shaft transitions from guidewire shaft sectional portion 106 to guidewire shaft distal portion 316, 416, 516. As shown in FIGS. 3-5 which are discussed in detail below, guidewire shaft distal portion 316, 416, 516 may be attached to, or enveloped within, distal portion 114 of catheter shaft 101. Guidewire shaft distal portion 316, 416, 516 extend for a relatively short distance with respect to the overall length of catheter 100 from essentially transition section 124 to distal tip 126. Guidewire shaft distal portion 316, 416, 516 include a guidewire lumen 318, 418, 518 respectively, such that the guidewire is within the guidewire lumen through distal portion 114 of catheter shaft 101 and balloon 120. The guidewire exits catheter 100 at distal tip 126.

Transition section 124 is located proximal to balloon 120. Transition section 124 is preferably located, but is not limited to, a distance of between 15 and 28 centimeters proximal of balloon 120. FIG. 3 is a sectional view along line A-A of FIG. 1, and illustrates an embodiment of transition section 124 of catheter shaft 101. FIG. 3 illustrates an inner surface 328 of inflation shaft 102, and an outer surface 334 of guidewire shaft 316. In this embodiment of the present invention, guidewire shaft 316 is disposed coaxially within inflation shaft 102, with an annular space between outer surface 334 of guidewire shaft 316 and inner surface 328 of inflation shaft 102 serving as a distal portion of inflation lumen 204 and being in fluid communication with balloon 120. This embodiment of balloon catheter 100 results in guidewire lumen 318 and inflation lumen 204 being in a coaxial arrangement in distal portion 114 of catheter shaft 101.

FIG. 4 is an alternate embodiment along line A-A of FIG. 1, and illustrates another embodiment of transition section 124 of catheter shaft 101. In this embodiment guidewire shaft 416 is disposed within inflation shaft 402 in a non-coaxial relationship. This alternate configuration results in guidewire lumen 418 and inflation lumen 204 being in a side-by-side arrangement in the distal portion 114 of catheter shaft 101. Other embodiments of balloon catheter 100 may include guidewire lumen 418 and inflation lumen 204 in other non-coaxial dual lumen arrangements, such as having a circular guidewire lumen above a D-shaped or crescent-shaped inflation lumen, as illustrated in FIG. 7.

FIG. 5 is an alternate embodiment along line A-A of FIG. 1, and illustrates another embodiment of transition section 124 of catheter 100. Alternatively, guidewire shaft 516 is shown attached to an outside surface 530 of a distal portion 502 of inflation shaft 102. This alternate configuration has a guidewire lumen 518 and a distal inflation lumen 504 disposed in a side-by-side relationship in distal portion 114 of catheter shaft 101.

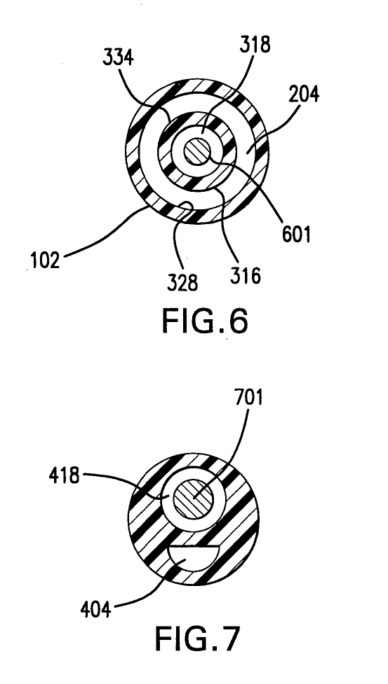

FIG. 6 is a cross-sectional view of distal portion 114 of catheter shaft 101 taken along line B-B of FIG. 1, and illustrates a coaxial dual lumen arrangement as discussed with reference to FIG. 3. As apparent in FIG. 6, inflation lumen 204 is formed between outer surface 334 of guidewire shaft 316 and inner surface 328 of inflation shaft 102 to allow inflation media to flow into balloon 120. FIG. 6 shows a guidewire 601 within guidewire lumen 318.

FIG. 7 is an alternate embodiment of distal portion 114 of catheter shaft 101 taken along line B-B of FIG. 1, and illustrates an extruded shaft having a non-coaxial arrangement of guidewire lumen 418 and inflation lumen 204, as discussed with reference to FIG. 4. Guidewire 701 is shown within guidewire lumen 418.

In one embodiment to form catheter 100 with guidewire shaft sectional portion 106, an appropriate length double lumen shaft is extruded. The cross-section of the double lumen catheter may vary, as shown in the exemplary cross-sections of the embodiments of FIGS. 2A, 2B, 2C and 2D. Using laser or blade cutting processes, the extruded shaft is machined to form guidewire shaft sectional portion 106. Segments of the extruded shaft are removed therefrom, forming proximal, distal and intermediate clips or segments of the guidewire shaft sectional portion as appropriate. The machined shaft, including guidewire shaft sectional portion 106, is then assembled with the remaining components of catheter 100 using catheter assembly techniques known in the art.

Inflation shaft 102, clips 108, 109, and 110, and guidewire shaft distal portion 316 are made of any appropriate polymeric material. Material choice depends on the application and performance requirements. Possible materials used in construction of inflation shaft 102 are polyethylene terephalate (PET), PEBAX, polypropylene, polyvinyl chloride, nylon, and polyethylene. In one embodiment, a proximal portion of inflation shaft 102 is formed from a reinforced polymeric tube or a hypotube. In another embodiment, inflation shaft 102 is extruded with a hypotube reinforcing a proximal length thereof.

Non-exhaustive examples of material for guidewire shaft 116 and guidewire shaft sectional portion 106 include polyethylene, PEBAX, nylon, TEFLON or combinations of any of these, either blended or co-extruded. Balloon 120 can be any appropriate shape or size, and any material, which is relatively elastic and deformable. Non-exhaustive examples for balloon 120 include polymers such as polyethylene, PEBAX, PET, nylon, and polyurethane. In addition, distal tip 126 can be braided with stainless steel or NITINOL wires to acquire the desired stiffness. The required tip stiffness and flexibility depends on the performance requirements.

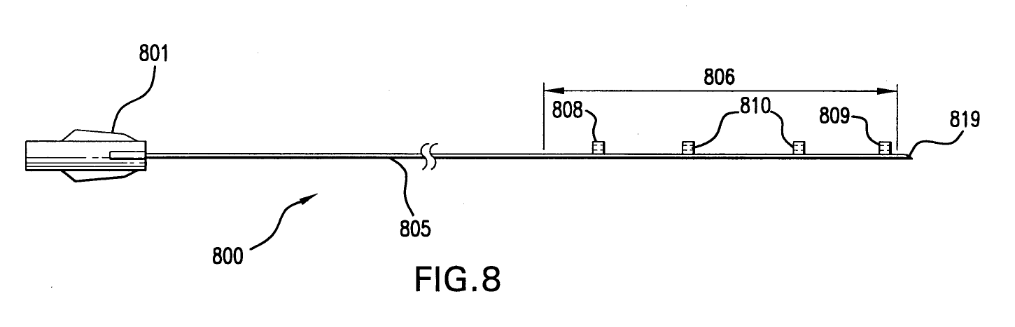

Another embodiment of the present invention is shown in FIG. 8, which is a side elevational view of an aspiration catheter 800 incorporating a sectional guidewire shaft 806 along a distal portion of an aspiration shaft 805. Aspiration shaft 805 includes an aspiration lumen (not shown) and is similar to other tubular members known in the art that are suitable for aspirating embolic or thrombotic matter from a vessel. Aspiration shaft 805 is a long, continuous tubular body with a cross-sectional diameter that is relatively large, typically with a diameter of from 0.7 mm to 18 mm. While the length of aspiration shaft 805 may vary depending upon the specific procedure, a typical length for aspiration shaft 805 is 145 cm.

A proximal aspiration port 801 is disposed at a proximal end of aspiration shaft 805. Proximal aspiration port 801 is adapted to be joined to a source of negative pressure, as is well-known in the art. For example, proximal aspiration port 801 may be a valve or a luer connector. The source of negative pressure may be a syringe or a line to a continuous vacuum source. Aspiration shaft 805 may be made from any of the materials as discussed above with reference to inflation shaft 102.

At a distal tip of aspiration catheter 800, aspiration shaft 805 includes a distal aspiration port 819. To increase the cross-sectional area of distal aspiration port 819 open to the vessel distal port 819 is set at an oblique angle to the rest of aspiration shaft 805. Further, the distal tip of catheter 800 may include a radiopaque marker (not shown) to aid in tracking the distal tip during the procedure. Such a radiopaque marker is typically a band of radiopaque material, such as platinum, fixedly attached to the distal tip of catheter 800.

As shown in FIG. 8, sectional guidewire shaft 806 is disposed substantially on the distal portion of aspiration shaft 805. Sectional guidewire shaft 806 includes clips 808, 809 and 810 which are positioned along an outer surface of aspiration shaft 805, or made integral therewith. Although four clips are shown, more or fewer clips maybe used as the application requires. Sectional guidewire shaft 806 is significantly shorter in length and has a significantly smaller lumen diameter than aspiration shaft 805. Sectional guidewire shaft 806 and clips 808, 809, and 810 are made of similar materials and in a similar manner as the various embodiments of sectional guidewire shaft 106 and clips 108, 109 and 110, shown and described above with reference to FIGS. 2 and 2A-2D. In the embodiment of FIG. 8, a guidewire is held along the distal portion of the aspiration catheter by the clips and is accessible to a clinician between the clips for ease of catheter exchange.

While this invention has been particularly shown and described with reference to preferred embodiments thereof, it will be understood by those skilled in the art that various changes in form and details may be made therein without departing from the spirit and scope of the invention.

1. A catheter comprising:
a first shaft having a proximal portion, a distal portion, and a first lumen;
a second shaft having a second lumen for a guidewire, said second shaft including a sectional portion comprised of a plurality of guidewire clips for holding the guidewire along the first shaft, wherein each of the guidewire clips is separated from an adjacent guidewire clip such that a clinician has access to the guidewire there between.

2. The catheter according to claim 1, wherein the plurality of clips includes a proximal clip, a distal clip and at least one intermediate clip disposed between the proximal and distal clips.

3. The catheter according to claim 2, wherein the at least one intermediate clip has an outer surface and a slit extending from the outer surface to the lumen thereof, such that the guidewire can be transversely loaded or unloaded through the slit.

4. The catheter according to claim 2, wherein there are at least two intermediate clips disposed between the proximal and distal clips

5. The catheter according to claim 1, wherein the catheter is used for angioplasty and said sectional portion of said second shaft is disposed along the proximal portion of said first shaft.

6. The catheter according to claim 1, wherein the catheter is an aspiration catheter and said sectional portion of said second shaft is disposed along the distal portion of said first shaft.

7. A balloon catheter comprising:
an inflation shaft having a proximal portion, a distal portion, and an inflation lumen;
a guidewire shaft having a guidewire lumen, wherein said guidewire shaft includes a proximal portion comprised of a plurality of clips wherein at least one of the clips is spaced from each adjacent clip in such a manner as to allow access to a guidewire proximally and distally thereof; and
a balloon disposed on the distal portion of the inflation shaft, wherein an interior of the balloon is in fluid communication with the inflation lumen.

8. The balloon catheter according to claim 7, wherein the plurality of clips includes a proximal clip, a distal clip and at least two intermediate clips disposed between the proximal and distal clips.

9. The catheter according to claim 8, wherein at least one of the intermediate clips has an outer surface and a slit extending from the outer surface to the guidewire lumen thereof, such that a guidewire can be transversely loaded or unloaded through the slit.

10. A stent delivery system comprising:
a balloon catheter, the balloon catheter including,
an inflation shaft having a proximal portion, a distal portion, and an inflation lumen;
a guidewire shaft having a guidewire lumen, a proximal sectional portion and a distal portion, wherein the proximal sectional portion of said guidewire shaft is further comprised of a plurality of short segments positioned along the proximal portion of said inflation shaft and the distal portion of said guidewire shaft is disposed within the distal portion of said inflation shaft; and
a balloon disposed on and in fluid communication with the distal portion of said inflation shaft; and
a stent mounted on said balloon.

11. The stent delivery system according to claim 10, wherein the plurality of short segments of the proximal sectional portion of said guidewire shaft includes a proximal segment, a distal segment and at least one intermediate segment disposed between the proximal and distal segments.

12. The stent delivery system according to claim 11, wherein the at least one intermediate segment includes a slit for ease of transversely loading and unloading a guidewire into and out of the guidewire lumen.

13. The stent delivery system according to claim 11, wherein the at least one intermediate segment is positioned between and separated from each of the proximal or distal segments such the guidewire is accessible proximally and distally of the intermediate segment.

14. An aspiration catheter comprising:
an aspiration shaft having a proximal portion, a distal portion, and an aspiration lumen; and
a guidewire shaft having a guidewire lumen, wherein said guidewire shaft is comprised of a sectional portion that includes a plurality of clips wherein at least one of the clips is spaced from each adjacent clip in such a manner as to allow access to a guidewire proximally and distally thereof, and wherein the sectional portion of said guidewire shaft is positioned along the distal portion of said aspiration shaft.

15. The balloon catheter according to claim 14, wherein the plurality of clips includes a proximal clip, a distal clip and at least two intermediate clips disposed between the proximal and distal clips.

16. The catheter according to claim 15, wherein at least one of the intermediate clips has an outer surface and a slit extending from the outer surface to the guidewire lumen thereof, such that a guidewire can be transversely loaded or unloaded through the slit.
